(19) United States
(12) Patent Application Publication
(10) Pub. No.: US 2023/0190972 A1
(43) Pub. Date: Jun. 22, 2023

(54) Method of Manufacture a Drinking Straw from Grass

(71) Applicants: Trongnghia D. Huynh, Cherry Hill, NJ (US); Hung T. Ha, Ho Chi Minh (VN); Mia L. Mai, Cherry Hill, NJ (US); Sang H. Tran, Milpitas, CA (US); Vinh H. Tran, San Jose, CA (US)

(72) Inventors: Trongnghia D. Huynh, Cherry Hill, NJ (US); Hung T. Ha, Ho Chi Minh (VN); Mia L. Mai, Cherry Hill, NJ (US); Sang H. Tran, Milpitas, CA (US); Vinh H. Tran, San Jose, CA (US)

(21) Appl. No.: 17/556,131

(22) Filed: Dec. 20, 2021

(51) Int. Cl. A61L 2/18; B08B 3/08

(52) U.S. Cl. CPC A61L 2/186; B08B 3/08; A47G 21/18

(57) ABSTRACT

The invention discloses a method for preparing straws from grass stems, which adopts a green and environmentally friendly method technology, takes the Lepironia articulata, known as grey sedge stems, baking soda, and hydrogen peroxide water as raw materials, and prepares the drinking straws under specific processing conditions. The method of the invention mainly comprises two aspects: firstly, the preparation of grass stems after being harvested from the field, and secondly, the process of turning grass stems into drinking straws is prepared from the membrane removal and cleaning processes using baking soda and hydrogen peroxide solution. According to the invention, the manufacturing processes are ecologically friendly and produce commercial straw products with zero waste, the technology disclosed by the invention has the advantages of environmental protection, low cost, and has great industrial application value.

### FIELD OF THE INVENTION

The invention belongs to the field of the process and manufacturing industry. More specifically, the invention pertains to manufacturing straws, and green, biodegradable, and zero-waste substitute manufacturing processes.

### BACKGROUND OF THE INVENTION

This invention relates to a process for the production of grass as an alternative for use as straws; these replacements employ green, biodegradable, and zero-waste Lepinoria grass instead of plastic.

As is known, plastic straws are usually produced by melting plastic resin such as HIPS, PVC, PE, PP, and ABS in form of pellets via extrusion at high temperatures. The normal production method is to subject the melted resin through several operations, so that a thin-wall tube is produced which is then subjected to a series of processes such as forming, coloring, and cutting. These manufacturing steps, which often consume a large amount of energy in terms of heat and electricity, can be greatly reduced in the new process of producing straws from grass. It is also known that in manufacturing plastic straws, the end product and wastes are plastics that are not biodegradable and cannot be broken down naturally by bacteria or other living things, and as a result, most of it ends up in our living habitats where it causes pollution through plastic wastes. These drawbacks are eliminated by the present invention.

The present invention is based on the discovery that the functionality of the drinking straw can be completed by the stem of Lepinoria grass. This invention, therefore, relates to a process for the manufacture of drinking straws from grass sources that do not have the shortcomings described. In addition, the new process consists principle of manufacturing processes that also consume less energy, friendly to the environment, and are more sustainable to the local region.

In carrying out the present invention, it is preferable to first process the grass stems to remove the foreign contaminations at room temperature with the oxygen-enhanced sanitizer, this also helps to remove the odor on the newly harvested grass stems.

The grass stems thus are soaked under slow oxygen released bath where they are treated and prepared for the cleaning process. Finally, part of the cleaned grass stems is transferred to the vacuum chamber where they are heat-treated at low temperature to remove moisture to further extend the product shelf-life; while others are dehydrated naturally at room temperature to preserve the color.

The important point about this new process is both final products and wastes are part of the grass sources meaning they are green, organic, and biodegradable. In addition, the material used in the manufacturing process can be sourced locally, and are sustainable and friendly to the environment.

Employing our invention, the manufacturing process of a drinking straw requires low, near room temperature conditions as a result the energy requirement of the production location is minimal which allows the site to be near the source and can be equipped with renewable energy sources which further reduce the carbon footprint and cost of transportation.

### DRAWINGS

The invention is better understood by reference to the following detailed description when read in conjunction with the appended drawing.

FIG. 1 is a photographic of the straw product in its ‘fresh form’ produced with the present invention.

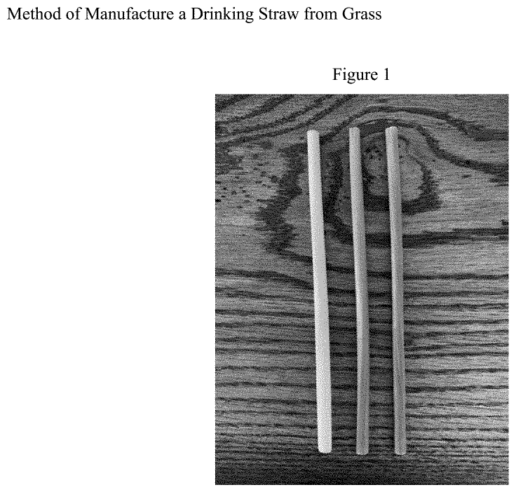

FIG. 2 is a photographic of the straw product in its ‘dry form’ produced with the present invention.

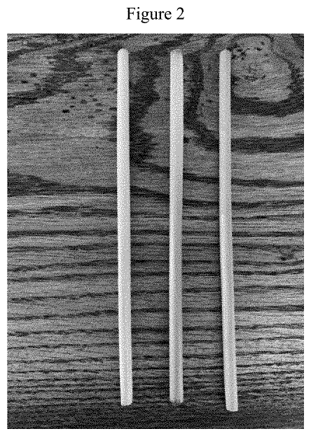

FIG. 3 is an embodiment of the manufacturing process of the grass straws, and shows a flow diagram representing the steps involved in the process of making the grass straws from grass stems with the present invention.

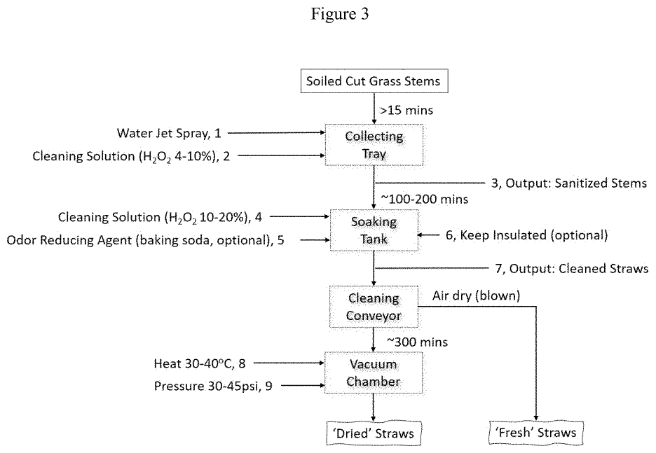

### DETAILED DESCRIPTION

In the first embodiment of the invention, a collecting stray is provided. The collecting stray is comprised of a cleaning means (2) to remove foreign contamination such as soil, dead leaves, and other undesirable matters introduced to or on already on the grass stems. In this embodiment of the invention, the water jet (1) or other liquid spraying nozzle is employed to discharge the cleaning solution (2) along the grass stem to physically remove soiled objects. Preferably, the cleaning solution used should contain a slow oxygen releaser to reduce the odor on the grass stem. In this certain embodiment, the sanitizing process should be between 5 to 10 minutes in time and should not last any longer than 15 minutes.

In an embodiment of the invention, sanitized stems (3) are placed in the cleaning tank to be soaked for an extended amount of time where the membrane inside softens and gets ready to be removed. In this embodiment of the invention, the soaking solution (4) used should contain the slow oxygen released agent and an activator agent (5) to boost the efficacy of the process.

The soaking time for the grass stems in this embodiment of the invention should be from 100 to 200 minutes and it is preferable that the tank is kept insulated (6).

The cleaned grass stems (7) are later moved to a conveyor belt for cleaning as stated in the next process which is optional. The cleaned steam, in this embodiment of the invention, is packed as ‘fresh straw’ or moved to the drying chamber where the stems are dried under heat and pressure to draw the moisture out. The prolonged heating process is kept under vacuumed environment and (8) at low temperature (9) to preserve the shape, color, and texture of the finish products.

1. A process for manufacturing drinking straws from grass as a sole material comprising the step of sanitizing said drinking straws, providing a cleaning operation to produce said drinking straws, and placing said drinking straws in a vacuum chamber while maintaining the temperature from about 25° C. to 45° C.

2. The process according to claim 1, wherein stems of said grass are sanitized in a temperature from about 20° C. to about 30° C.

3. The process according to claim 2, wherein the grass stems are sanitized in a concentration of an oxygen-based solution which is maintained from about 4% to 10% in volume.

4. The process according to claim 2, wherein the grass stems are sanitized in an oxygen-based solution which is used until all the contaminants are removed but the sanitization process cannot be longer than 15 minutes.

5. The process according to claim 1, wherein stems of said grass are soaked in a cleaning solution during the cleaning process and the temperature of the solution bath is maintained at about from 35° C. to 45° C. wherein no external heat is applied.

6. The process according to claim 5, wherein the grass stems are soaked in a concentration of an oxidation solution from about 15% to about 20% in volume.

7. The process according to claim 6, wherein the grass stems are soaked in the oxidation solution from about 100 minutes to 200 minutes.

8. The process according to claim 1, wherein stems of said grass are thereafter dried in the vacuum chamber with a pressure maintained at about 30 psi to 45 psi.

9. The process according to claim 8, wherein the grass stems are thereafter dried in the vacuum chamber, the temperature is maintained at about 30° C. to 45° C.

10. The process according to claim 8, wherein the grass stems are thereafter dried in the vacuum chamber, the duration of the drying process is kept from about 180 minutes to about 300 minutes.
